(19) United States
(12) Patent Application Publication
(10) Pub. No.: US 2023/0399366 A1
(43) Pub. Date: Dec. 14, 2023

(54) CREATING A BIOMIMETIC VEGAN VERSION OF COLLAGEN USING PLANT-BASED AMINO ACIDS AND OPTIONAL INDUCTORS TO EMULATE THE BODY'S NATURAL COLLAGEN BUILDING PROCESS FOR NUTRACEUTICAL AND COSMECEUTICAL PURPOSES

(71) Applicant: Corpowell BV, Schilde (BE)

(72) Inventor: Anthony Van Campen, Halle (BE)

(21) Appl. No.: 18/036,275

(22) PCT Filed: Nov. 10, 2021
(86) PCT No.: PCT/IB2021/060415

§ 371 Date: May 10, 2023

(30) Foreign Application Priority Data
Nov. 11, 2020 (ZA) 2020/07000

(51) Int. Cl. C07K 14/415

(52) U.S. Cl. CPC C07K 14/415

(57) ABSTRACT

A plant-based alternative to animal-sourced collagen offering a sustainable, animal-friendly option for use in nutraceuticals and cosmeceuticals by creating a biomimetic of the human collagen (by mimicking its unique amino acid profile) and optionally adding inductors to promote the human collagen production.

### CROSS-REFERENCE TO RELATED APPLICATIONS

The present application is the National Phase entry of International Patent Application No. PCT/IB2021/060415 filed Nov. 10, 2021, which claims priority to South African Patent Application No. 2020/07000 filed Nov. 11, 2020, the entire contents of which are hereby incorporated by reference into this application.

### TECHNICAL FIELD

The main protein in the human body is collagen, a fibrous molecule consisting of a specific amino acid profile in a characteristic triple helix structure that forms the conjunctive and connective tissues in the human body, essentially skin, joints, and bones.

### BACKGROUND

Aging is a natural process which involves changes in collagen and elastin expression. Aging negatively affects the connective tissues in both the skin and the cartilage due to reduced collagen production by the human body.

To reverse or slow down these aging effects animal-sourced collagen is taken in an oral supplement form to encourage the body to use it to create more of its own collagen or is applied topically to penetrate the skin barrier and stimulate the production of collagen.

Hydrolysed collagen, as a nutraceutical (biologically active compound) supplement, has been extensively shown to benefit human skin and cartilage connective tissues. It has been demonstrated that C14-labeled collagen peptides can reach the skin, cartilage, bones, and muscles and remain in these tissues up to 14 days after a single ingestion.

The current approach to creating nutraceuticals and cosmetics is to use animal-sourced collagen from various sources such as bovine, porcine, chicken or marine sources.

To improve the biological availability of the collagen found in these animal sources, the obtained collagen (ex from bovine hides) is hydrolysed to create smaller molecules (“peptides”-small chains of amino acids) that allow the body to digest easier or allow the transdermal passage.

The positive effects of animal collagen seem to be obtained, in part, by the supply of amino acids from the digestion of collagen. The amino acids in collagen have a unique ratio, characterised for example by high glycine contents.

The human body uses these amino acids to build its own collagen in a complex process.

Furthermore, some collagen peptides seem to pass the digestive track and exert a messenger function positively impacting their target tissue (ex skin, cartilage, . . . )

In summary: currently animal-sourced collagen, in hydrolysed or pure form (such as in bone broth) is ingested or topically applied to stimulate the human body to produce more native collagen.

### SUMMARY

The present disclosure aims to emulate the natural synthesis process for collagen using vegan analogues. This is accomplished by mapping out the human collagen amino acid profile and creating a biomimetic using plant-based amino acid sources to exactly replicate this human amino acid profile.

The human body is optionally further stimulated by plant based inductors to use these amino acids as building blocks to naturally produce the collagen sequence and triple helix structure characteristic to collagen using vegan sources.

### BRIEF DESCRIPTION OF THE DRAWINGS

FIG. 1 schematically shows a diagram according to the present disclosure.

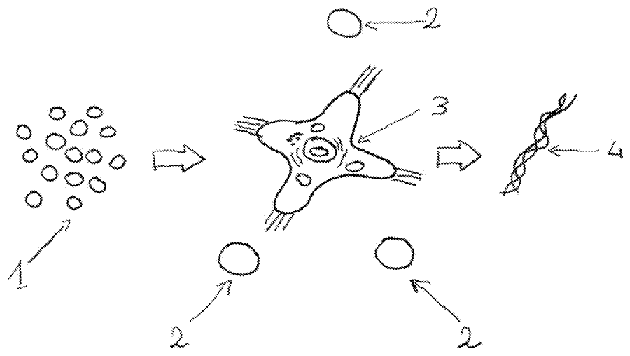

### DETAILED DESCRIPTION

The first step in the present disclosure is to determine the exact amino acid profile of the targeted collagen type to be mimicked.

There are more than twenty types of collagen in the human body all with their own unique amino acid profile and corresponding characteristics. To create a valid biomimetic alternative using vegan analogues it is extremely important that the exact amino acid profile of the targeted collagen is replicated.

This can be done through a variety of methods including but not limited to methods such as Biomdrin based on machine learning (www.biomdrin.com). Some amino acid profiles can be found in previous scientific research as well (example https://www.ncbi.nlm.nih.gov/) or in scientific databases.

To come to a usable formula and amino acid profile that can be replicated using vegan amino acids, the amino acid profiles of different helixes of a collagen molecule need to be mapped out. For a first product VeCollal® under this new innovative process we have mapped out the exact amino acid profile of Human type 1 collagen.

This was done using a machine learning datatool called Biomdrin. This enables us to describe the α1 and α2 collagen chain amino acid sequence and establish the amino acid content in a 2:1 ratio.

To create the commercially available product VeCollal® this process has been applied to type 1 collagen and the exact amino acid profile has been returned as follows in Table 1.

TABLE 1
Amino Acid Profile of Type 1 Collagen
Amino Acids on Human Type I Collagen

| | Three | One | | | | | | | Human | Human |
|---|---|---|---|---|---|---|---|---|---|---|
| Amino | Letter | Letter | | Alpha [symbol] | | Alpha [symbol] | | Alpha [symbol] | Type [symbol] | Type [symbol] |
| Acid | Symbol | Symbol | Alpha [symbol] | [%] | Alpha [symbol] | [%] | Alpha [symbol] | [%] | Collagen | Collagen [%] |
| Glycine | Gly | G | [symbol] | [symbol] | [symbol] | [symbol] | [symbol] | [symbol] | [symbol] | [symbol] |
| Pro [symbol] | Pro | P | [symbol] | [symbol] | [symbol] | [symbol] | [symbol] | [symbol] | [symbol] | [symbol] |
| [symbol] | Ala | [symbol] | [symbol] | [symbol] | [symbol] | [symbol] | [symbol] | [symbol] | [symbol] | [symbol] |
| Glutamic acid | Glu | E | 75 | [symbol] | 75 | [symbol] | 66 | [symbol] | [symbol] | [symbol] |
| Arginine | Arg | [symbol] | [symbol] | [symbol] | [symbol] | [symbol] | [symbol] | [symbol] | [symbol] | [symbol] |
| [symbol] acid | Asp | O | 66 | [symbol] | 66 | [symbol] | [symbol] | [symbol] | [symbol] | [symbol] |
| Serine | Ser | [symbol] | [symbol] | [symbol] | [symbol] | [symbol] | [symbol] | [symbol] | [symbol] | [symbol] |
| Lysine | Lys | K | [symbol] | [symbol] | [symbol] | [symbol] | [symbol] | [symbol] | [symbol] | [symbol] |
| Leucine | Leu | [symbol] | [symbol] | [symbol] | [symbol] | [symbol] | [symbol] | [symbol] | [symbol] | [symbol] |
| Valine | Val | [symbol] | 47 | [symbol] | 47 | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| Glutamine | Gln | [symbol] | 49 | [symbol] | 49 | [symbol] | 33 | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | 24 | [symbol] | 24 | [symbol] | 32 | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | C | [symbol] | [symbol] | [symbol] | [symbol] | [symbol] | [symbol] | [symbol] | [symbol] |
| [symbol] | [symbol] | [symbol] | [symbol] | [symbol] | [symbol] | 0.3 | [symbol] | [symbol] | [symbol] | [symbol] |
| [symbol] | [symbol] | M | [symbol] | [symbol] | [symbol] | 0.3 | [symbol] | [symbol] | 36 | [symbol] |
| [symbol] | [symbol] | H | 9 | [symbol] | 9 | [symbol] | [symbol] | [symbol] | [symbol] | [symbol] |
| Tryptophan | Trp | W | 6 | [symbol] | 6 | [symbol] | [symbol] | [symbol] | 17 | [symbol] |

[symbol]

indicates data missing or illegible when filed

The same process is applicable to any other collagen type available in the human body. For example the same process could easily be applied to type 3 human collagen and falls under the same patent application.

Once the exact amino acid profile has been determined the biomimetic or collagen analogue can be constructed using individual free form amino acids of a vegan origin or a combination of vegan protein sources with compatible amino acid profiles or a combination of both methods;

Most vegan amino acids are derived from a fermentation process of molasses, sugar cane, corn or other plants to be classed as vegan and are commercially available from several suppliers such as Ajinomoto.

A collagen biomimetic by definition has the exact same amino acid profile as the collagen type it is aiming to mimic.

The next step in our process is to optionally supply the human body with inductors aiming at increasing the collagen production by stimulation of the fibroblasts.

Our first commercial development VeCollal® has inductors such as vitamin C, asiaticoside and ginseng as seen in the formulation below, table 2.

TABLE 2
DETERMINATION OF RATIO AMINO
ACIDS TO DIFFERENT INDUCTORS

| Component | Min. | | Max. | | % |
|---|---|---|---|---|---|
| Vegan Amino Acids | 100 | g | 120 | g | 99.957-85.516 |
| Vitamin C | 17.612 | mg | 20 | g | 0.0176-14.253 |
| Ginseng | 0.1 | mg | 25 | mg | 0.0001-0.018 |
| Asiaticoside | 25 | mg | 300 | mg | 0.025-0.214 |

But all other inductors, active ingredients, herbs, botanicals or other compounds aiming at stimulating the collagen production of the human body after or while ingesting or topically applying a collagen biomimetic by timulating the fibroblasts would fall under this scope.

The complete process aims at supplying the body with a biomimetic supply of amino acids mimicking a certain collagen type, orally or transdermal, and optionally stimulating the human body with inductors aimed at increasing the collagen production through fibroblast stimulation
1. amino acids
   The amino acids supplied need to be the exact amino acid profile of human collagen to be considered a biomimetic. To supply the human body with a biomimetic collagen alternative it is essential that's the exact ratio of amino acids is supplied as found in the human collagen type that is emulated. This can be achieved with different methods such but not limited to the use of Biomdrin to determine the exact ratios and then achieving these ratios in the final product using free from amino acids, vegan protein sources (example: pea protein) blends or a combination hereof.
2. The effect of the biomimetic amino acid profile can be enhanced by optionally adding inductors to the product (nutraceutical or cosmeceutical). The inductors have the characteristics of stimulating human collagen production through different pathways such as but not limited to stimulation of the fibroblast.
3. The fibroblasts are responsible for the production of collagen and will use the amino acids and optional inductors provided to produce human collagen.
4. The end product of the process is human collagen, produced by the body, of different possible types such as but not limited to type 1.

The formats of this product for commercialisation include but are not limited to:
Powders
Liquids
Gummies
Softgels
Granules
Sprays
Face creams
Tablets
Gels
Injections

1. A vegan, plant-based alternative to animal-sourced collagen offering a sustainable, animal-friendly option for use in nutraceuticals and cosmeceuticals aiming at providing the same health and cosmetic benefits as obtained by animal collagen, wherein it mimics the targeted human collagen type by exactly copying its amino acid profile (“biomimicking”) and optionally adding inductors that will stimulate the human body to use these amino acids to produce native collagen by stimulating the fibroblasts and aims at providing the benefits of collagen without using any animal sources.
